(19) United States
(12) Patent Application Publication
(10) Pub. No.: US 2023/0395212 A1
(43) Pub. Date: Dec. 7, 2023

(54) DISTRIBUTED SYSTEM FOR LOGIC-DRIVEN DATA ROUTING

(71) Applicant: FAMULUS HEALTH LLC, Charleston, SC (US)

(72) Inventor: Michael J. Szwajkos, Bluffton, SC (US)

(73) Assignee: FAMULUS HEALTH LLC, Charleston, SC (US)

(21) Appl. No.: 18/203,479

(22) Filed: May 30, 2023

Related U.S. Application Data
(60) Provisional application No. 63/348,266, filed on Jun. 2, 2022.

(51) Int. Cl. G16H 10/60; G16H 20/10

(52) U.S. Cl. CPC G16H 10/60; G16H 20/10

(57) ABSTRACT

Systems, computer program products, and methods are described herein for logic-driven data routing. The present invention is configured to receive, from a user input device, a request to process a transaction; retrieve information associated with the request; identify one or more third party devices based on at least the information associated with the request; broadcast the request simultaneously to the one or more third party devices; receive, from the one or more third party devices, one or more responses to the request; implement, using a decisioning subsystem, one or more conditional parameters on the one or more responses; determine an optimal response based on at least implementing the one or more conditional parameters on the one or more responses; and transmit control signals configured to cause the user input device to display the optimal response.

### CROSS-REFERENCE TO RELATED APPLICATIONS

This application claims the benefit of priority from U.S. Provisional Patent Application No. 63/348,266, filed on Jun. 2, 2022, the contents of which are hereby incorporated by reference in its entirety.

### FIELD OF THE INVENTION

The present invention embraces a distributed system for logic-driven data routing.

### BACKGROUND

Typically, when a user who is a healthcare plan beneficiary brings a prescription into a pharmacy to be filled, the pharmacist reviews the information in the prescription and begins the fulfillment process. The pharmacist enters the information in the prescription, including beneficiary's name, medication details, and Issuer Identification Number (IIN), and submits a fulfillment request on the user's behalf. When the pharmacist submits the request, the information is transmitted to a switch operator that directs the information to the correct Pharmacy Benefits Manager (PBM) network. Then, the PBM then determines a payment amount based on the user's healthcare plan's coverage, including the plan's required copay or coinsurance requirement, any deductible that may need to be satisfied, as well as any prior authorization or program requirement that must be met. The PBM then transmits this information to a switch operator that routes the information back to the pharmacy.

Depending on the type of insurance plan, the payment amount for the same medication may vary across different networks. It is not uncommon for a beneficiary with insurance plan IP_1 may have to pay more for the same medication than a beneficiary with insurance plan IP_2. This is due to the fact that when a prescription fulfillment request is submitted, it is routed directly to the PBM network identified by the IIN and is beholden to the payment amount that is defined thereby, even though other plans, such as national discount pharmacy networks, or national funded networks may have lower pricing options. Therefore, there is a need for a pre-adjudication platform for implementing logic based decisioning and routing for pricing prescription medication to obtain the lowest pricing from not only the PBM network identified by the IIN, but also other additional networks.

Applicant has identified a number of deficiencies and problems associated with logic-driven data routing. Through applied effort, ingenuity, and innovation, many of these identified problems have been solved by developing solutions that are included in embodiments of the present disclosure, many examples of which are described in detail herein.

### SUMMARY

The following presents a simplified summary of one or more embodiments of the present invention, in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments of the present invention in a simplified form as a prelude to the more detailed description that is presented later.

In one aspect, a system for logic-driven data routing is presented. The system comprising: a processing device; a non-transitory storage device containing instructions when executed by the processing device, causes the processing device to: receive, from a user input device, a request to process a transaction; retrieve information associated with the request; identify one or more third party devices based on at least the information associated with the request; broadcast the request simultaneously to the one or more third party devices; receive, from the one or more third party devices, one or more responses to the request; implement, using a decisioning subsystem, one or more conditional parameters on the one or more responses; determine an optimal response based on at least implementing the one or more conditional parameters on the one or more responses; and transmit control signals configured to cause the user input device to display the optimal response.

In some embodiments, the information associated with the request comprises information associated with a patient, information associated with a prescription, and information associated with a benefits provider.

In some embodiments, the request comprises a request for pricing medications listed in the prescription.

In some embodiments, the response comprises cost of the medications listed in the prescription.

In some embodiments, executing the instructions further causes the processing device to: route, using routing logic, the request to a first third party device based on at least the information associated with the benefits provider.

In some embodiments, the first third party device comprises at least a Pharmacy Benefits Manager (PBM) network associated with the benefits provider.

In some embodiments, executing the instructions further causes the processing device to: determine, using program eligibility logic, whether the user is eligible to receive benefits provided by additional networks; and route, using the routing logic, the request to the first third party device and a second third party device in an instance in which the user is eligible to receive the benefits provided by the additional networks.

In some embodiments, the second third party device comprises at least discount networks, direct retail discount networks, PBM discount networks, direct retail funded networks, PBM funded networks, and/or mid-market PBM funded network.

In some embodiments, executing the instructions further causes the processing device to: determine that the request is in a non-standardized format; convert, using a proprietary application installed on the user input device, the request from the non-standardized format to a standardized format; and broadcast the request in the standardized format to the one or more third party devices.

In another aspect, a computer program product for logic-driven data routing is presented. The computer program product comprising a non-transitory computer-readable medium comprising code causing an apparatus to: receive, from a user input device, a request to process a transaction; retrieve information associated with the request; identify one or more third party devices based on at least the information associated with the request; broadcast the request simultaneously to the one or more third party devices; receive, from the one or more third party devices, one or more responses to the request; implement, using a decisioning subsystem, one or more conditional parameters on the one or more responses; determine an optimal response based on at least implementing the one or more conditional parameters on the one or more responses; and transmit control signals configured to cause the user input device to display the optimal response.

In yet another aspect, a method for logic-driven data routing is presented. The method comprising: receiving, from a user input device, a request to process a transaction; retrieving information associated with the request; identifying one or more third party devices based on at least the information associated with the request; broadcasting the request simultaneously to the one or more third party devices; receiving, from the one or more third party devices, one or more responses to the request; implementing, using a decisioning subsystem, one or more conditional parameters on the one or more responses; determining an optimal response based on at least implementing the one or more conditional parameters on the one or more responses; and transmitting control signals configured to cause the user input device to display the optimal response.

The features, functions, and advantages that have been discussed may be achieved independently in various embodiments of the present invention or may be combined with yet other embodiments, further details of which can be seen with reference to the following description and drawings.

### BRIEF DESCRIPTION OF THE DRAWINGS

Having thus described embodiments of the invention in general terms, reference will now be made the accompanying drawings, wherein:

FIGS. 1A-1C illustrates technical components of an exemplary distributed computing environment for pre-adjudication platform for implementing logic based decisioning and routing of transactions, in accordance with an embodiment of the invention;

FIG. 2 illustrates a process flow for pre-adjudication platform for implementing logic based decisioning and routing of transactions, in accordance with an embodiment of the invention; and

FIG. 3 illustrates a data flow diagram for implementing logic based decisioning and routing for pricing prescription medication, in accordance with an embodiment of the invention.

### DETAILED DESCRIPTION OF EMBODIMENTS OF THE INVENTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout.

As used herein, an “entity” may be any organization employing information technology resources and particularly technology infrastructure configured for processing data. Typically, these data can be related to the people who work for the organization, its products or services, the customers, or any other aspect of the operations of the organization. As such, the entity may be any institution, group, association, establishment, company, union, authority, or the like, employing information technology resources for processing data.

As described herein, a “user” may be an individual submitting a request to the entity. As such, in some embodiments, the user may be an individual having past relationships, current relationships or potential future relationships with an entity.

As used herein, “authentication credentials” may be any information that can be used to identify of a user. For example, a system may prompt a user to enter authentication information such as a username, a password, a personal identification number (PIN), a passcode, biometric information (e.g., iris recognition, retina scans, fingerprints, finger veins, palm veins, palm prints, digital bone anatomy/structure and positioning (distal phalanges, intermediate phalanges, proximal phalanges, and the like), an answer to a security question, a unique intrinsic user activity, such as making a predefined motion with a user device. This authentication information may be used to authenticate the identity of the user (e.g., determine that the authentication information is associated with the account) and determine that the user has authority to access an account or system. In some embodiments, the system may be owned or operated by an entity. In such embodiments, the entity may employ additional computer systems, such as authentication servers, to validate and certify resources inputted by the plurality of users within the system. The system may further use its authentication servers to certify the identity of users of the system, such that other users may verify the identity of the certified users. In some embodiments, the entity may certify the identity of the users. Furthermore, authentication information or permission may be assigned to or required from a user, application, computing node, computing cluster, or the like to access stored data within at least a portion of the system.

As used herein, a “user interface” may be a point of human-computer interaction and communication in a device that allows a user to input information, such as commands or data, into a device, or that allows the device to output information to the user. For example, the user interface includes a graphical user interface (GUI) or an interface to input computer-executable instructions that direct a processor to carry out specific functions. The user interface typically employs certain input and output devices such as a display, mouse, keyboard, button, touchpad, touch screen, microphone, speaker, LED, light, joystick, switch, buzzer, bell, and/or other user input/output device for communicating with one or more users.

It should also be understood that “operatively coupled,” as used herein, means that the components may be formed integrally with each other, or may be formed separately and coupled together. Furthermore, “operatively coupled” means that the components may be formed directly to each other, or to each other with one or more components located between the components that are operatively coupled together. Furthermore, “operatively coupled” may mean that the components are detachable from each other, or that they are permanently coupled together. Furthermore, operatively coupled components may mean that the components retain at least some freedom of movement in one or more directions or may be rotated about an axis (i.e., rotationally coupled, pivotally coupled). Furthermore, “operatively coupled” may mean that components may be electronically connected and/or in fluid communication with one another.

As used herein, an “interaction” may refer to any communication between one or more users, one or more entities or institutions, one or more devices, nodes, clusters, or systems within the distributed computing environment described herein. For example, an interaction may refer to a transfer of data between devices, an accessing of stored data by one or more nodes of a computing cluster, a transmission of a requested task, or the like.

It should be understood that the word “exemplary” is used herein to mean “serving as an example, instance, or illustration.” Any implementation described herein as “exemplary” is not necessarily to be construed as advantageous over other implementations.

As used herein, “determining” may encompass a variety of actions. For example, “determining” may include calculating, computing, processing, deriving, investigating, ascertaining, and/or the like. Furthermore, “determining” may also include receiving (e.g., receiving information), accessing (e.g., accessing data in a memory), and/or the like. Also, “determining” may include resolving, selecting, choosing, calculating, establishing, and/or the like. Determining may also include ascertaining that a parameter matches a predetermined criterion, including that a threshold has been met, passed, exceeded, and so on.

Among other reasons, expanded healthcare coverage has increased the volume of prescriptions being fulfilled by pharmacies. Most pharmacies use a prescription routing and switching partner to parse the prescription information and route the prescription to the appropriate PBM network. PBMs are typically entities that are independent of the benefit provider, e.g., an insurance company, and contract with the benefit provider to process claims, part of which involves negotiating payment amounts for medications for pharmacy benefits. However, depending on the type of insurance plan and/or benefits provider, the payment amount for the same medication may vary. This is due to the fact that when a prescription fulfillment request is submitted, it is routed directly to the PBM network that is associated with the IIN and is beholden to the payment amount that is defined by the plan only, even though other plans, such as national discount pharmacy networks, or national funded networks may have lower pricing options.

Embodiments of the present invention implements a pre-adjudication platform in real-time that is capable of providing essential healthcare connectivity among retail pharmacies, physicians, health plans, pharmacy benefit managers, government agencies and/or pharmaceutical manufacturers. To this end, the embodiments of the present invention receive a high volume of requests (e.g., B1/B2 claim transactions, or other supported formats) from multiple pharmacies at the same time. In response, the present invention communicates with multiple edge devices, i.e., end-point devices associated with various networks (including PBM networks and various additional networks) to communicate the requests and subsequently receive responses therefrom. In one example, the requests may include a price-point for a particular prescription medication. Then, the present invention implements routing logic, program eligibility logic, and/or conditional parameters to process each response to determine the optimal response for each request. In a related example, the optimal response may be the lowest price for the particular prescription medication. These optimal responses are then routed back to the pharmacy that originated the corresponding request. As such, embodiments of the present invention have the ability to handle the requests as they originated from the pharmacies, and then respond with the appropriate response for the pharmacies to track and reconcile the requests efficiently.

Embodiments of the invention manage and interpret large volumes of data from multiple sources, each with its unique structure and format. This includes information about insurance plans, PBMs, national discount pharmacy networks, and national funded networks, as well as medication data. To handle such data, embodiments of the invention implement robust and efficient data processing elements such as data integration, data cleaning, data transformation, data storage, and/or the like. With an increase in the volume of prescriptions, the embodiments of the invention handle thousands and potentially millions of transaction requests simultaneously from multiple pharmacies, providers, and payer institutions. By implementing routing logic, the present invention identifies, accurately, specific partner networks to route each request to for processing. Also, embodiments of the invention execute advance rule-setting mechanism to evaluate the various pricing options and determine the lowest cost for a given medication. These rules are customizable and flexible, as they consider various factors such as the type of medication, the specifics of the insurance plan, and current prices across different networks. As such, embodiments of the invention execute logic and conditional parameters on each response to determine the optimal response for each request. Furthermore, embodiments of the invention effectively interface with different networks, retrieve pricing information, compare the prices, and then direct the request to the network that offers the lowest price. Accordingly, the present invention uses existing computing resources, such as processing resources, storage resources, network resources, and/or the like more efficiently, thus implementing the solution with fewer steps. Furthermore, by implementing conditional parameters on each response to determine the optimal response, the present invention provides a more accurate solution each time, thus reducing the number of resources required to remedy any errors made due to a less accurate response. Lastly, the present invention removes manual input and waste from the implementation of the solution, thus not only improving speed and efficiency of the process and conserving computing resources, but also improving user interaction experience. Furthermore, the technical solution described herein uses a rigorous, computerized process to perform specific tasks and/or activities that were not previously performed. In specific implementations, the technical solution bypasses a series of steps previously implemented, thus further conserving computing resources.

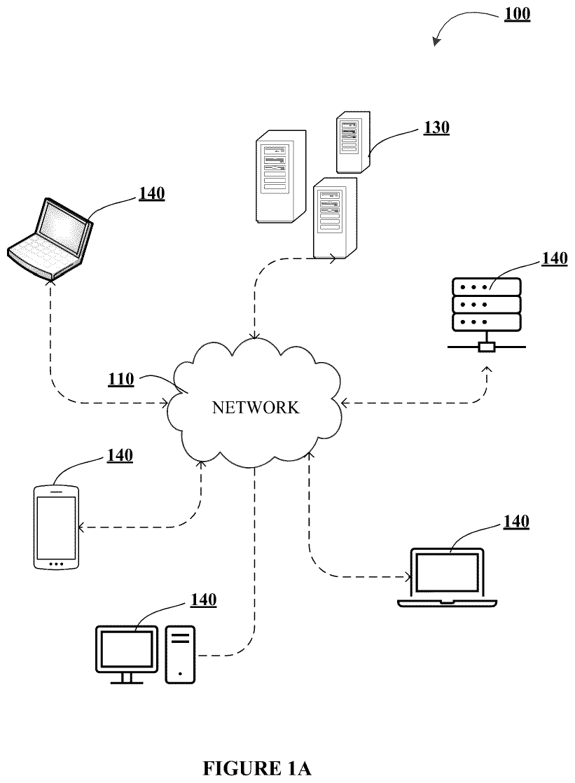

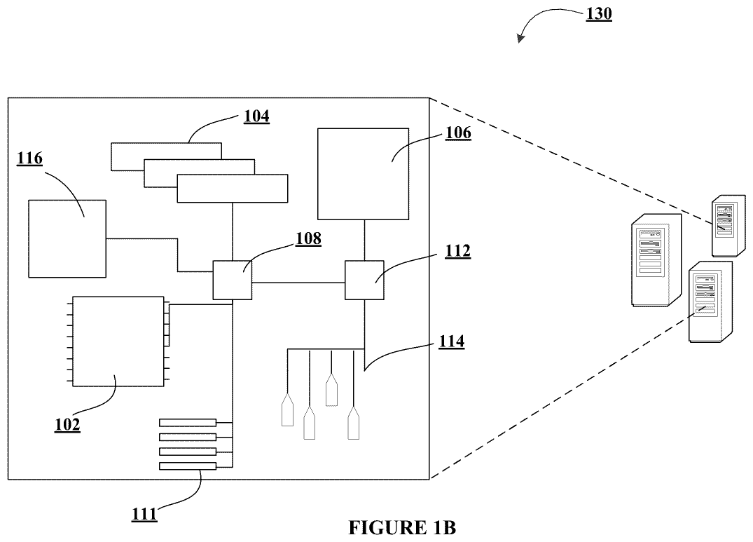

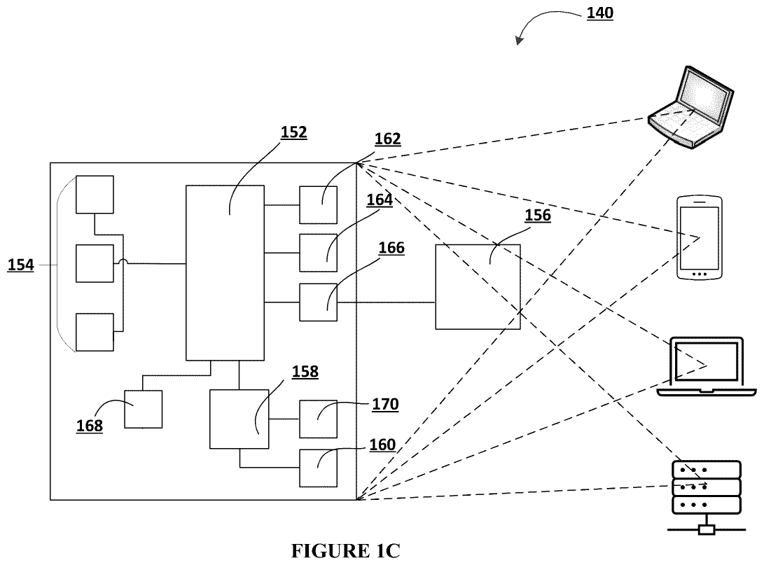

FIGS. 1A-IC illustrate technical components of an exemplary distributed computing environment for a pre-adjudication platform for implementing logic based decisioning and routing of transactions 100, in accordance with an embodiment of the invention. As shown in FIG. 1A, the distributed computing environment 100 contemplated herein may include a system 130, end-point device(s) 140, and a network 110 over which the system 130 and end-point device(s) 140 communicate therebetween. FIG. 1A illustrates only one example of an embodiment of the distributed computing environment 100, and it will be appreciated that in other embodiments one or more of the systems, devices, and/or servers may be combined into a single system, device, or server, or be made up of multiple systems, devices, or servers. Also, the distributed computing environment 100 may include multiple systems, same or similar to system 130, with each system providing portions of the necessary operations (e.g., as a server bank, a group of blade servers, or a multi-processor system).

In some embodiments, the system 130 and the end-point device(s) 140 may have a client-server relationship in which the end-point device(s) 140 are remote devices that request and receive service from a centralized server, i.e., the system 130. In some other embodiments, the system 130 and the end-point device(s) 140 may have a peer-to-peer relationship in which the system 130 and the end-point device(s) 140 are considered equal and all have the same abilities to use the resources available on the network 110. Instead of having a central server (e.g., system 130) which would act as the shared drive, each device that is connect to the network 110 would act as the server for the files stored on it.

The system 130 may represent various forms of servers, such as web servers, database servers, file server, or the like, various forms of digital computing devices, such as laptops, desktops, workstations, or the like, or any other auxiliary network devices, such as wearable devices, Internet-of-things devices, electronic kiosk devices, mainframes, or the like, or any combination of the aforementioned.

The end-point device(s) 140 may represent various forms of electronic devices, including user input devices such as personal digital assistants, cellular telephones, smartphones, laptops, desktops, electronic payment kiosks, and/or the like, edge devices such as routers, routing switches, integrated access devices (IAD), and/or the like, workstations, or any end-point that may be used as an entry and/or an exit point to access data from external networks. For purposes of this invention, end-point device(s) 140 may represent pharmacies, switch providers, edge devices for pharmacy benefit managers (PBM) networks and additional networks such as large national discount networks, direct retail discount networks, large PBM discount networks, direct retail funded networks, large PBM funded network, mid-market PBM funded network, and/or the like.

In examples where the end-point device(s) 140 is a pharmacy, a user, such as a consumer, pharmacist, or other pharmacy employee, may utilize the end-point device(s) 140 in preparing and inputting a prescription drug request or order for processing, and subsequently retrieve or otherwise receive data, including payment (including discount) information for the prescription request or order. In examples where the end-point device(s) 140 is a switch provider, requests received from the pharmacy related to pharmacy, benefits, and/or discount transactions is routed via the switch provider for processing, and subsequent responses are provided to the switch provider to be routed back to the pharmacy. In examples where the end-point device(s) 140 is an edge device associated with PBM network, requests related to pharmacy, benefits, pricing, and/or discount transactions received from the pharmacy, and/or the switch provider may be received, processed, and fulfilled by the PBM network. Similarly, in examples where the end-point device(s) 140 is an edge device associated with the additional networks, requests related to pharmacy, benefits, pricing, and/or discount transactions received from the pharmacy, the switch provider, and/or the pharmaceutical manufacturer may be received, processed, and fulfilled by the additional networks.

The network 110 may be a distributed network that is spread over different networks. This provides a single data communication network, which can be managed jointly or separately by each network. Besides shared communication within the network, the distributed network often also supports distributed processing. The network 110 may be a form of digital communication network such as a telecommunication network, a local area network (“LAN”), a wide area network (“WAN”), a global area network (“GAN”), the Internet, or any combination of the foregoing. The network 110 may be secure and/or unsecure and may also include wireless and/or wired and/or optical interconnection technology.

It is to be understood that the structure of the distributed computing environment and its components, connections and relationships, and their functions, are meant to be exemplary only, and are not meant to limit implementations of the inventions described and/or claimed in this document. In one example, the distributed computing environment 100 may include more, fewer, or different components. In another example, some or all of the portions of the distributed computing environment 100 may be combined into a single portion or all of the portions of the system 130 may be separated into two or more distinct portions.

FIG. 1B illustrates an exemplary component-level structure of the system 130, in accordance with an embodiment of the invention. As shown in FIG. 1B, the system 130 may include a processor 102, memory 104, input/output (I/O) device 116, and a storage device 110. The system 130 may also include a high-speed interface 108 connecting to the memory 104, and a low-speed interface 112 connecting to low speed bus 114 and storage device 110. Each of the components 102, 104, 108, 110, and 112 may be operatively coupled to one another using various buses and may be mounted on a common motherboard or in other manners as appropriate. As described herein, the processor 102 may include a number of subsystems to execute the portions of processes described herein. Each subsystem may be a self-contained component of a larger system (e.g., system 130) and capable of being configured to execute specialized processes as part of the larger system.

The processor 102 can process instructions, such as instructions of an application that may perform the functions disclosed herein. These instructions may be stored in the memory 104 (e.g., non-transitory storage device) or on the storage device 110, for execution within the system 130 using any subsystems described herein. It is to be understood that the system 130 may use, as appropriate, multiple processors, along with multiple memories, and/or I/O devices, to execute the processes described herein.

The memory 104 stores information within the system 130. In one implementation, the memory 104 is a volatile memory unit or units, such as volatile random access memory (RAM) having a cache area for the temporary storage of information, such as a command, a current operating state of the distributed computing environment 100, an intended operating state of the distributed computing environment 100, instructions related to various methods and/or functionalities described herein, and/or the like. In another implementation, the memory 104 is a non-volatile memory unit or units. The memory 104 may also be another form of computer-readable medium, such as a magnetic or optical disk, which may be embedded and/or may be removable. The non-volatile memory may additionally or alternatively include an EEPROM, flash memory, and/or the like for storage of information such as instructions and/or data that may be read during execution of computer instructions. The memory 104 may store, recall, receive, transmit, and/or access various files and/or information used by the system 130 during operation.

The storage device 106 is capable of providing mass storage for the system 130. In one aspect, the storage device 106 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier may be a non-transitory computer- or machine-readable storage medium, such as the memory 104, the storage device 104, or memory on processor 102.

The high-speed interface 108 manages bandwidth-intensive operations for the system 130, while the low speed controller 112 manages lower bandwidth-intensive operations. Such allocation of functions is exemplary only. In some embodiments, the high-speed interface 108 is coupled to memory 104, input/output (I/O) device 116 (e.g., through a graphics processor or accelerator), and to high-speed expansion ports 111, which may accept various expansion cards (not shown). In such an implementation, low-speed controller 112 is coupled to storage device 106 and low-speed expansion port 114. The low-speed expansion port 114, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The system 130 may be implemented in a number of different forms. For example, it may be implemented as a standard server, or multiple times in a group of such servers. Additionally, the system 130 may also be implemented as part of a rack server system or a personal computer such as a laptop computer. Alternatively, components from system 130 may be combined with one or more other same or similar systems and an entire system 130 may be made up of multiple computing devices communicating with each other.

FIG. 1C illustrates an exemplary component-level structure of the end-point device(s) 140, in accordance with an embodiment of the invention. As shown in FIG. 1C, the end-point device(s) 140 includes a processor 152, memory 154, an input/output device such as a display 156, a communication interface 158, and a transceiver 160, among other components. The end-point device(s) 140 may also be provided with a storage device, such as a microdrive or other device, to provide additional storage. Each of the components 152, 154, 158, and 160, are interconnected using various buses, and several of the components may be mounted on a common motherboard or in other manners as appropriate.

The processor 152 is configured to execute instructions within the end-point device(s) 140, including instructions stored in the memory 154, which in one embodiment includes the instructions of an application that may perform the functions disclosed herein, including certain logic, data processing, and data storing functions. The processor may be implemented as a chipset of chips that include separate and multiple analog and digital processors. The processor may be configured to provide, for example, for coordination of the other components of the end-point device(s) 140, such as control of user interfaces, applications run by end-point device(s) 140, and wireless communication by end-point device(s) 140.

The processor 152 may be configured to communicate with the user through control interface 164 and display interface 166 coupled to a display 156. The display 156 may be, for example, a TFT LCD (Thin-Film-Transistor Liquid Crystal Display) or an OLED (Organic Light Emitting Diode) display, or other appropriate display technology. The display interface 156 may comprise appropriate circuitry and configured for driving the display 156 to present graphical and other information to a user. The control interface 164 may receive commands from a user and convert them for submission to the processor 152. In addition, an external interface 168 may be provided in communication with processor 152, so as to enable near area communication of end-point device(s) 140 with other devices. External interface 168 may provide, for example, for wired communication in some implementations, or for wireless communication in other implementations, and multiple interfaces may also be used.

The memory 154 stores information within the end-point device(s) 140. The memory 154 can be implemented as one or more of a computer-readable medium or media, a volatile memory unit or units, or a non-volatile memory unit or units. Expansion memory may also be provided and connected to end-point device(s) 140 through an expansion interface (not shown), which may include, for example, a SIMM (Single In Line Memory Module) card interface. Such expansion memory may provide extra storage space for end-point device(s) 140 or may also store applications or other information therein. In some embodiments, expansion memory may include instructions to carry out or supplement the processes described above and may include secure information also. For example, expansion memory may be provided as a security module for end-point device(s) 140 and may be programmed with instructions that permit secure use of end-point device(s) 140. In addition, secure applications may be provided via the SIMM cards, along with additional information, such as placing identifying information on the SIMM card in a non-hackable manner.

The memory 154 may include, for example, flash memory and/or NVRAM memory. In one aspect, a computer program product is tangibly embodied in an information carrier. The computer program product contains instructions that, when executed, perform one or more methods, such as those described herein. The information carrier is a computer- or machine-readable medium, such as the memory 154, expansion memory, memory on processor 152, or a propagated signal that may be received, for example, over transceiver 160 or external interface 168.

In some embodiments, the user may use the end-point device(s) 140 to transmit and/or receive information or commands to and from the system 130 via the network 110. Any communication between the system 130 and the end-point device(s) 140 may be subject to an authentication protocol allowing the system 130 to maintain security by permitting only authenticated users (or processes) to access the protected resources of the system 130, which may include servers, databases, applications, and/or any of the components described herein. To this end, the system 130 may trigger an authentication subsystem that may require the user (or process) to provide authentication credentials to determine whether the user (or process) is eligible to access the protected resources. Once the authentication credentials are validated and the user (or process) is authenticated, the authentication subsystem may provide the user (or process) with permissioned access to the protected resources. Similarly, the end-point device(s) 140 may provide the system 130 (or other client devices) permissioned access to the protected resources of the end-point device(s) 140, which may include a GPS device, an image capturing component (e.g., camera), a microphone, and/or a speaker.

The end-point device(s) 140 may communicate with the system 130 through communication interface 158, which may include digital signal processing circuitry where necessary. Communication interface 158 may provide for communications under various modes or protocols, such as the Internet Protocol (IP) suite (commonly known as TCP/IP). Protocols in the IP suite define end-to-end data handling methods for everything from packetizing, addressing and routing, to receiving. Broken down into layers, the IP suite includes the link layer, containing communication methods for data that remains within a single network segment (link); the Internet layer, providing internetworking between independent networks; the transport layer, handling host-to-host communication; and the application layer, providing process-to-process data exchange for applications. Each layer contains a stack of protocols used for communications. In addition, the communication interface 158 may provide for communications under various telecommunications standards (2G, 3G, 4G, 5G, and/or the like) using their respective layered protocol stacks. These communications may occur through a transceiver 160, such as radio-frequency transceiver. In addition, short-range communication may occur, such as using a Bluetooth, Wi-Fi, or other such transceiver (not shown). In addition, GPS (Global Positioning System) receiver module 170 may provide additional navigation—and location-related wireless data to end-point device(s) 140, which may be used as appropriate by applications running thereon, and in some embodiments, one or more applications operating on the system 130.

The end-point device(s) 140 may also communicate audibly using audio codec 162, which may receive spoken information from a user and convert it to usable digital information. Audio codec 162 may likewise generate audible sound for a user, such as through a speaker, e.g., in a handset of end-point device(s) 140. Such sound may include sound from voice telephone calls, may include recorded sound (e.g., voice messages, music files, etc.) and may also include sound generated by one or more applications operating on the end-point device(s) 140, and in some embodiments, one or more applications operating on the system 130.

Various implementations of the distributed computing environment 100, including the system 130 and end-point device(s) 140, and techniques described here can be realized in digital electronic circuitry, integrated circuitry, specially designed ASICs (application specific integrated circuits), computer hardware, firmware, software, and/or combinations thereof.

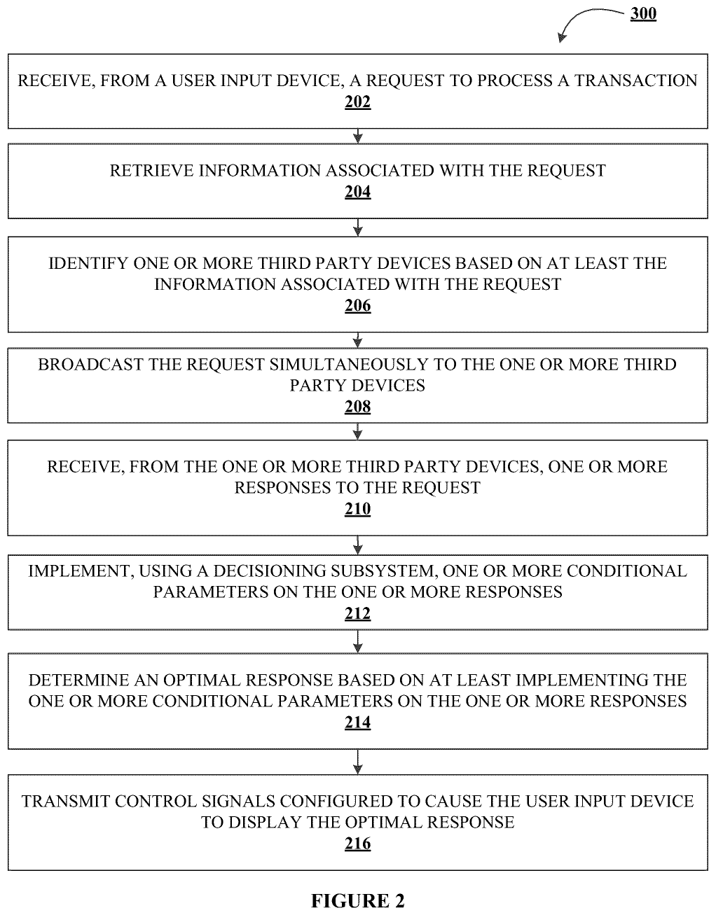

FIG. 2 illustrates a process flow for pre-adjudication platform for implementing logic based decisioning and routing of transactions, in accordance with an embodiment of the invention. As shown in block 202, the process flow includes receiving, from a user input device, a request to process a transaction. As described herein, a user (e.g., patient) may initiate the request by approaching a pharmacy. At the pharmacy's location, the user may present the necessary documentation, such as prescription for dispensing of the medication, to the pharmacist to initiate the request. If the user is part of a healthcare plan provided by a benefits provider (e.g., insurance company), then the user typically has a prescription medication benefit through the benefits provider. In such cases, the user may also present the name of the benefits provider, including specific health plan or information identifying the specific PBM associated with the benefits provider.

The information provided by the user is then input by the pharmacist into an end-point device (e.g., a user input device) located at the pharmacy to generate the request. In some embodiments, the system may be configured to provide for installation, a proprietary application for installation on the user input device located at the pharmacy. When a new request is to be submitted, the pharmacist may open a portal using this proprietary application on the user input device, and input the information provided by the user. In one aspect, the proprietary application may be configured to re-structure the information provided by the pharmacist into a predefined standard set by organizations such as National Council for Prescription Drug Programs (NCPDP), American National Standards Institute (ANSI), or the like, or any other predefined standard with a formatted structure.

In embodiments where a switch partner serves as an intermediary for routing claims, the system may be configured to receive the request from the user input device via the switch partner. In embodiments where the user input device is capable of communicating directly with the system using the proprietary application, the system may be configured to receive the request directly from the user input device.

Next, as shown in block 204, the process flow includes retrieving information associated with the request. In some embodiments, the information associated with the request may include, among other elements, information associated with the user (e.g., personal information), information associated with the prescription (e.g., medication and associated dosage), or the information associated with the benefits provider, such as an Issuer Identification Number (IIN/BIN), and/or Processor Control Number (PCN). If the benefits provider uses the NCPDP Pharmacy ID, the IIN is displayed on the user's card. The system may then be configured to use this IIN/PCN number to route the request, using routing logic, the right destination, i.e., PBM network associated with the benefits provider.

Next, as shown in block 206, the process flow includes identifying one or more third party devices based on at least the information associated with the request. In some embodiments, the third party devices may be associated with one or more networks, such as the PBM network, capable of processing the request. The IIN/PCN number may be used to identify the PBM network, to whom the request is to be routed for processing.

Next, as shown in block 208, the process flow includes broadcasting the request simultaneously to the one or more third party devices. In some embodiments, prior to broadcasting the request simultaneously to all the third party devices, the system may be configured to determine, using program eligibility logic, whether the user is eligible to receive the benefits provided by the additional networks. If the user is ineligible, the system may be configured to route the request only to the PBM network associated with the benefits provider for processing. If the user is eligible, the system may be configured to route the request to the PBM network associated with the benefits provider and the additional networks simultaneously. In one aspect, the additional networks may include large discount networks, direct retail discount networks, large PBM discount networks, direct retail funded networks, large PBM funded network, mid-market PBM funded network, and/or the like. Each additional network may be associated with a PBM who is independent of that network and is contracted with that network to process requests. Similar to the PBM network associated with the benefits provider, the PBM associated with each additional network processes request in accordance with one or more parameters (e.g., medication payment amounts) specific to that network. In other words, should a request be routed to direct retail funded network (say), the request will be processed by the PBM associated with the direct retail funded network in accordance with one or more parameters specific to the direct retail funded network.

Next, as shown in block 210, the process flow includes receiving, from the one or more third party devices, one or more responses to the request. In some embodiments, the response may include a negotiated payment amount for the medications. It is not uncommon for different networks to be associated with different payment amounts for the same medication. In one aspect, the system may be configured to continuously monitor each additional network to periodically receive an updated list of medications with their corresponding payment amounts. In response, the system may be configured to store the updated list of medications with their corresponding payment amounts in a repository. When a request is received, instead of routing the claim to each additional network, the system may be configured to retrieve the updated list of medications with their corresponding payment amounts for each additional network to determine a preliminary response to the request. In another aspect, the system may be configured to establish a communication link with third party devices associated with each additional network to retrieve their updated list of medications and corresponding payment amounts in each time a request is received.

Next, as shown in block 212, the process flow includes implementing, using a decisioning subsystem, one or more conditional parameters on the one or more responses. In some embodiments, the decisioning subsystem may be configured to implement one or more conditional parameters on the one or more responses received from each network (including the PBM network). In one aspect, the one or more conditional parameters may include determining the cheapest payment amount for each medication listed in the prescription across various networks.

Next, as shown in block 214, the process flow includes determining an optimal response based on at least implementing the one or more conditional parameters on the one or more responses. First, the system may be configured to determine whether the PBM network associated with the benefits provider has the cheapest payment amount for each medication listed in the prescription. In this regard, the system may be configured to compare the payment amounts for the medications from each network and determine the network that has the least payment amount for each medication. For medications where the PBM network has the cheapest payment amount, the optimal response may identify the PBM network associated with the benefits provider and list the payment amounts proposed by the PBM network deemed to be the cheapest payment amounts for that medication. The request is then processed, i.e., adjudicated, by the PBM. For medications where the one of the additional networks has the cheapest payment amount, the optimal response may identify the network and the list the payment amounts proposed by the network deemed to be the cheapest payment amounts for that medication. The request is then routed to the PBM associated with that network for processing, i.e., adjudication.

Next, as shown in block 216, the process flow includes transmitting control signals configured to cause the user input device to display the optimal response. By implementing conditional parameters on the responses received from the networks, the system may be configured to determine the cheapest payment amount for each medication for the user in the optimal response.

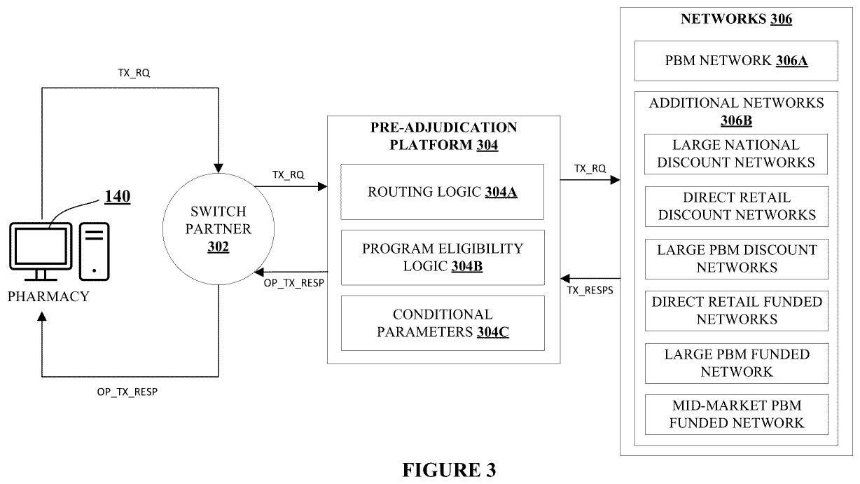

FIG. 3 illustrates a data flow diagram for implementing logic based decisioning and routing for pricing prescription medication 300, in accordance with an embodiment of the invention. As shown in FIG. 3, the pharmacy 140 may transmit a request TX_RQ to a switch partner 302. The switch partner 302 may in turn transmit the request TX_RQ to a pre-adjudication platform 304. The pre-adjudication platform 304 may include at least routing logic 304A, program eligibility logic 304B, and conditional parameters 304C. The pre-adjudication platform 304 may be implement routing logic 304A on the request TX_RQ to route the request to the one or more networks 306 for processing. Prior to broadcasting the request, the pre-adjudication platform 304 may implement program eligibility logic to determine whether the user is eligible to have their request broadcasted to the additional networks 306B in addition to the PBM network 306A, i.e., the PBM associated with the benefits provider. If the user is ineligible, the pre-adjudication platform 304 may route, using the routing logic 304A, the request only to the PBM network 306A. If the user is eligible, the pre-adjudication platform 304 may route, using the routing logic 304A, the request to the PBM network 306A and the additional networks 306B simultaneously.

Each network, i.e., the PBM network 306A and the additional networks 306B, may transmit their responses TX_RESPS to the pre-adjudication platform 304. The pre-adjudication platform 304 may then implement conditional parameters 304C on the responses TX_RESPS to determine an optimal response OP_TX_RESP. As described herein, the TX_RESPS may include predetermined payment amounts for medications and the OP_TX_RESP may be the lowest payment amount for the medications.

While the pre-adjudication platform 304 is described here to route transactions associated with transaction type B1—a transaction used to request payment from the processor (e.g., benefits provider) for a user (e.g., patient) for claims billed according to appropriate healthcare plan parameters, i.e., pricing prescription medication, it may also be used to route other transaction types, including B2, B3, E1, N1, and/or the like. A B2 transaction is a transaction that is used to cancel a claim that has been processed previously, i.e., previously paid B1 claim reversal. When the pre-adjudication platform 304 receives a B2 request, it may determine, using routing logic 306A, whether the associated B1 request was processed by the pre-adjudication platform 304. If the B1 request was not processed by the pre-adjudication platform 304, it may be routed, using routing logic 306A, to the PBM network 306A for additional processing, i.e., claim reversal. On the other hand, if the B1 request was processed by the pre-adjudication platform 304, then the pre-adjudication platform 304 may determine whether the optimal payment amount determined for the B1 request was based on the payment amount provided by the PBM network 306A or one of the additional networks 306B. If the optimal amount was provided by the PBM network 306A, the pre-adjudication platform 304 may route, using routing logic 306A, the B2 request to the PBM network socket for additional processing. On the other hand, if the optimal amount was provided by one of the additional networks 306B, the pre-adjudication platform 304 may route the B2 request to the specific additional network 306B that provided the payment amount determined to be optimal for additional processing. Once the additional processing is completed, i.e., the B1 claim reversed, the pre-adjudication platform may then transmit information to the pharmacy 140 stating as such, including any remittance information arising from the claim reversal.

In some embodiments, for eligible users, the system may be configured to generate a personalized dashboard providing information about savings that user has accrued over time by using the pre-adjudication platform 304. In some embodiments, the pre-adjudication platform 304 may initiate a dashboard script configured to generate a dynamic display interface with graphical representation of the information for the user.

In some embodiments, the system may be configured to integrate directly with the healthcare plans provided by the networks such that the user is always provided with the benefit of lowest price for medication, without any additional eligibility membership requirements.

In some embodiments, the system may be configured to provide eligible members automated access to all non-covered medications, including NCPDP rejects codes.

In some embodiments, the system may be configured to capture the manufacture copay savings during the adjudication process to lower out of pockets costs for eligible members and healthcare plans' pharmacy spend.

In some embodiments, the system may be configured to integrate manufacturer rebates into the payment amounts provided by the PBM partner directly at the point-of-sale (POS) of the pharmacy.

In some embodiments, the system may be configured to provide and manage a web portal for healthcare professionals (e.g., physicians) to facilitate the handling of medical claims, where they can be converted to the pharmacy benefit and adjudicated efficiently.

In some embodiments, the system may be configured to serve as a central hub partner for various networks, pharmacies, manufacturers, etc., to monitor and manage specialty prescriptions for healthcare plans.

As will be appreciated by one of ordinary skill in the art, the present invention may be embodied as an apparatus (including, for example, a system, a machine, a device, a computer program product, and/or the like), as a method (including, for example, a business process, a computer-implemented process, and/or the like), or as any combination of the foregoing. Accordingly, embodiments of the present invention may take the form of an entirely software embodiment (including firmware, resident software, micro-code, and the like), an entirely hardware embodiment, or an embodiment combining software and hardware aspects that may generally be referred to herein as a “system.” Furthermore, embodiments of the present invention may take the form of a computer program product that includes a computer-readable storage medium having computer-executable program code portions stored therein. As used herein, a processor may be “configured to” perform a certain function in a variety of ways, including, for example, by having one or more special-purpose circuits perform the functions by executing one or more computer-executable program code portions embodied in a computer-readable medium, and/or having one or more application-specific circuits perform the function.

It will be understood that any suitable computer-readable medium may be utilized. The computer-readable medium may include, but is not limited to, a non-transitory computer-readable medium, such as a tangible electronic, magnetic, optical, infrared, electromagnetic, and/or semiconductor system, apparatus, and/or device. For example, in some embodiments, the non-transitory computer-readable medium includes a tangible medium such as a portable computer diskette, a hard disk, a random access memory (RAM), a read-only memory (ROM), an erasable programmable read-only memory (EPROM or Flash memory), a compact disc read-only memory (CD-ROM), and/or some other tangible optical and/or magnetic storage device. In other embodiments of the present invention, however, the computer-readable medium may be transitory, such as a propagation signal including computer-executable program code portions embodied therein.

It will also be understood that one or more computer-executable program code portions for carrying out the specialized operations of the present invention may be required on the specialized computer include object-oriented, scripted, and/or unscripted programming languages, such as, for example, Java, Perl, Smalltalk, C++, SAS, SQL, Python, Objective C, and/or the like. In some embodiments, the one or more computer-executable program code portions for carrying out operations of embodiments of the present invention are written in conventional procedural programming languages, such as the “C” programming languages and/or similar programming languages. The computer program code may alternatively or additionally be written in one or more multi-paradigm programming languages, such as, for example, F #.

It will further be understood that some embodiments of the present invention are described herein with reference to flowchart illustrations and/or block diagrams of systems, methods, and/or computer program products. It will be understood that each block included in the flowchart illustrations and/or block diagrams, and combinations of blocks included in the flowchart illustrations and/or block diagrams, may be implemented by one or more computer-executable program code portions. These computer-executable program code portions execute via the processor of the computer and/or other programmable data processing apparatus and create mechanisms for implementing the steps and/or functions represented by the flowchart(s) and/or block diagram block(s).

It will also be understood that the one or more computer-executable program code portions may be stored in a transitory or non-transitory computer-readable medium (e.g., a memory, and the like) that can direct a computer and/or other programmable data processing apparatus to function in a particular manner, such that the computer-executable program code portions stored in the computer-readable medium produce an article of manufacture, including instruction mechanisms which implement the steps and/or functions specified in the flowchart(s) and/or block diagram block(s).

The one or more computer-executable program code portions may also be loaded onto a computer and/or other programmable data processing apparatus to cause a series of operational steps to be performed on the computer and/or other programmable apparatus. In some embodiments, this produces a computer-implemented process such that the one or more computer-executable program code portions which execute on the computer and/or other programmable apparatus provide operational steps to implement the steps specified in the flowchart(s) and/or the functions specified in the block diagram block(s). Alternatively, computer-implemented steps may be combined with operator and/or human-implemented steps in order to carry out an embodiment of the present invention.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A system for logic-driven data routing, the system comprising:
a processing device;
a non-transitory storage device containing instructions when executed by the processing device, causes the processing device to:
receive, from a user input device, a request to process a transaction;
retrieve information associated with the request;
identify one or more third party devices based on at least the information associated with the request;
broadcast the request simultaneously to the one or more third party devices;
receive, from the one or more third party devices, one or more responses to the request;
implement, using a decisioning subsystem, one or more conditional parameters on the one or more responses;
determine an optimal response based on at least implementing the one or more conditional parameters on the one or more responses; and
transmit control signals configured to cause the user input device to display the optimal response.

2. The system of claim 1, wherein the information associated with the request comprises information associated with a patient, information associated with a prescription, and information associated with a benefits provider.

3. The system of claim 2, wherein the request comprises a request for pricing medications listed in the prescription.

4. The system of claim 3, wherein the response comprises cost of the medications listed in the prescription.

5. The system of claim 2, wherein executing the instructions further causes the processing device to:
route, using routing logic, the request to a first third party device based on at least the information associated with the benefits provider.

6. The system of claim 5, wherein the first third party device comprises at least a Pharmacy Benefits Manager (PBM) network associated with the benefits provider.

7. The system of claim 6, wherein executing the instructions further causes the processing device to:
determine, using program eligibility logic, whether the user is eligible to receive benefits provided by additional networks; and
route, using the routing logic, the request to the first third party device and a second third party device in an instance in which the user is eligible to receive the benefits provided by the additional networks.

8. The system of claim 7, wherein the second third party device comprises at least discount networks, direct retail discount networks, PBM discount networks, direct retail funded networks, PBM funded networks, and/or mid-market PBM funded network.

9. The system of claim 1, wherein executing the instructions further causes the processing device to:
determine that the request is in a non-standardized format;
convert, using a proprietary application installed on the user input device, the request from the non-standardized format to a standardized format; and
broadcast the request in the standardized format to the one or more third party devices.

10. A computer program product for logic-driven data routing, the computer program product comprising a non-transitory computer-readable medium comprising code causing an apparatus to:
receive, from a user input device, a request to process a transaction;
retrieve information associated with the request;
identify one or more third party devices based on at least the information associated with the request;
broadcast the request simultaneously to the one or more third party devices;
receive, from the one or more third party devices, one or more responses to the request;
implement, using a decisioning subsystem, one or more conditional parameters on the one or more responses;
determine an optimal response based on at least implementing the one or more conditional parameters on the one or more responses; and
transmit control signals configured to cause the user input device to display the optimal response.

11. The computer program product of claim 10, wherein the information associated with the request comprises information associated with a patient, information associated with a prescription, and information associated with a benefits provider.

12. The computer program product of claim 11, wherein the request comprises a request for pricing medications listed in the prescription.

13. The computer program product of claim 12, wherein the response comprises cost of the medications listed in the prescription.

14. The computer program product of claim 11, wherein the code further causes the apparatus to:
route, using routing logic, the request to a first third party device based on at least the information associated with the benefits provider.

15. The computer program product of claim 14, wherein the first third party device comprises at least a Pharmacy Benefits Manager (PBM) network associated with the benefits provider.

16. The computer program product of claim 15, wherein the code further causes the apparatus to:
determine, using program eligibility logic, whether the user is eligible to receive benefits provided by additional networks; and
route, using the routing logic, the request to the first third party device and a second third party device in an instance in which the user is eligible to receive the benefits provided by the additional networks.

17. The computer program product of claim 16, wherein the second third party device comprises at least discount networks, direct retail discount networks, PBM discount networks, direct retail funded networks, PBM funded networks, and/or mid-market PBM funded network.

18. The computer program product of claim 10, wherein the code further causes the apparatus to:
determine that the request is in a non-standardized format;
convert, using a proprietary application installed on the user input device, the request from the non-standardized format to a standardized format; and
broadcast the request in the standardized format to the one or more third party devices.

19. A method for logic-driven data routing, the method comprising:
receiving, from a user input device, a request to process a transaction;
retrieving information associated with the request;
identifying one or more third party devices based on at least the information associated with the request;
broadcasting the request simultaneously to the one or more third party devices;
receiving, from the one or more third party devices, one or more responses to the request;
implementing, using a decisioning subsystem, one or more conditional parameters on the one or more responses;
determining an optimal response based on at least implementing the one or more conditional parameters on the one or more responses; and
transmitting control signals configured to cause the user input device to display the optimal response.

20. The method of claim 19, wherein the method further comprises:
determining that the request is in a non-standardized format;
converting, using a proprietary application installed on the user input device, the request from the non-standardized format to a standardized format; and
broadcasting the request in the standardized format to the one or more third party devices.
